(19) United States
(12) Patent Application Publication
(10) Pub. No.: US 2005/0027133 A1
(43) Pub. Date: Feb. 3, 2005

(54) Method of making a catalyst for direct oxidation of an alkene to an alkene oxide

(75) Inventors: Patricia A. Hooks, Houston, TX (US); David L. Sullivan, Houston, TX (US); Michael D. Mier, Waller, TX (US); Jaap W. van Hal, Fresno, TX (US); Xiankuan Zhang, Houston, TX (US)

Correspondence Address: Jim Wheelington;SABIC Americas, Inc., SABIC Technology Center, 1600 Industrial Blvd., Sugar Land, TX, 77478, US

(73) Assignee: Saudi Basic Industries Corporation

(21) Appl. No.: 10/741,385

(22) Filed: Dec. 19, 2003

Related U.S. Application Data
(60) Provisional application No. 60/490,693, filed on Jul. 29, 2003.

(51) Int. Cl. C07D 301/03

(52) U.S. Cl. 549/535

(57) ABSTRACT

This invention is for a method of preparing a supported silver-containing catalyst for use in a process of making an alkene oxide from an alkene and an oxygen-containing gas by oxidation of the alkene to the corresponding epoxide. The catalyst contains metallic silver on a support. The catalyst may also contain small amount of compounds of metals or halides as promoters to improve selectivity, activity, conversion, stability or yield. The catalyst is prepared by forming a slurry of a silver compound, water and one or more organic compounds having at least one functional group of the formula —NX₂, —OX or [(═O) (—OX)] wherein X is hydrogen or an alkyl of one to three carbon atoms, X being the same or different, and wherein at least one functional group is bound to a terminal carbon. This slurry is formed before contact with the support material to deposit silver on the support.

### BACKGROUND OF THE INVENTION

1. Field of the Invention

This invention relates to a method of preparing a supported silver-containing catalyst for use in a process of making an alkene oxide from an alkene and an oxygen-containing gas by oxidation of the alkene to the corresponding epoxide.

2. Description of the Prior Art

Producing an alkene oxide or epoxide, particularly propylene oxide, by direct oxidation of an alkene in the presence of a silver-containing catalyst is well known. The reaction ideally proceeds as follows:
2 CH₃CH═CH₂+O₂-------->2 CH₃CHOCH₂
but CO₂ and H₂O can be produced as byproducts.

The catalyst generally contains metallic silver on a support. The catalyst may also contain small amount of alkali metals such as potassium, sodium, rubidium or cesium as promoters to improve selectivity, activity, conversion, stability or yield.

There are several methods in the prior art for preparing silver-containing catalysts for direct oxidation of an alkene, particularly propylene, to an alkene oxide, particularly propylene oxide.

U.S. Pat. No. 6,399,794 discloses a preparation of a catalyst for direct epoxidation of olefins, such as propylene, which includes forming a reaction slurry of a titanium silicate, a palladium compound and deionized water. The catalyst may be used for epoxidation in the liquid phase, gas phase or supercritical phase, but preferably in the liquid phase. Oxygen and hydrogen are required in the epoxidation process using this catalyst and an inert carrier gas is preferably used.

U.S. Pat. No. 6,392,066 discloses the deposition of silver onto a carrier in the preparation of a catalyst for propylene epoxidation. Several techniques for silver deposition are disclosed including impregnation of the support with a silver solution and precipitation of the silver into a slurry in which the support is placed for deposition of the silver on the support from the slurry during heating to remove the liquids. The working examples appear to use a precipitation technique.

U.S. Pat. No. 5,965,480 discloses a method for introducing silver and/or a promoter to a support to form a catalyst for the vapor phase oxidation of propylene to propylene oxide by an impregnation process in which a silver compound is dissolved in a solvent to form a solution in which the support is immersed to form a pasty mixture or slurry that is subsequently heated to dry the solid, remove volatiles and reduce the silver. Silver chloride may be applied to the support in the form of a suspension, slurry or dry solid.

U.S. Pat. No. 5,864,047 discloses forming a supported silver catalyst for vapor phase oxidation of propylene to propylene oxide by combining the support material with a silver compound solution which may optionally include a rhenium compound and potassium salt to form a coating paste or slurry with a alkaline earth metal compound which is dried and calcined.

U.S. Pat. No. 5,861,519 discloses catalysts for direct oxidation of propylene to propylene oxide. A catalyst preparation procedure in the working examples (Example 4) included forming a slurry of a calcined catalyst and a potassium nitrate solution.

U.S. Pat. No. 5,780,657 discloses catalysts for direct oxidation of propylene to propylene oxide. The methods of introducing silver to a support includes forming a solution of a silver compound in which a support is immersed to form a pasty mixture or slurry. If a promoting amount of silver chloride is used, it may be in a suspension or a slurry or as a dry solid to apply the silver to the support.

Catalyst performance is usually described by “conversion”, “selectivity”, “catalyst life” and “productivity”. “Conversion” is the percentage of alkene which is reacted. “Selectivity” is the percentage of reacted alkene which is converted to alkene oxide. “Catalyst life” is the longevity or useful life of the catalyst. “Productivity” is the amount of alkene oxide produced per amount of catalyst over a time unit, e.g. pounds of propylene oxide per cubic foot of catalyst per hour (lbs PO/ft³ catalyst-hr). For satisfactory performance, each of these factors should be considered. These factors may have a relationship between and among each other so that an increase in one may result in a decrease in another. Catalyst performance may be affected by catalyst composition, method of making the catalyst and process variation in use of the catalyst.

### SUMMARY OF THE INVENTION

The invention is for a process for making a catalyst used for oxidation of an alkene, such as propylene, to an alkene oxide, such as propylene oxide. The catalyst is made by:
a) forming a slurry of a silver compound, water and one or more organic compound(s) having at least one functional group of the formula —NX₂, —OX or [(═O) (—OX)] wherein X is hydrogen or an alkyl of one to three carbon atoms, X being the same or different, and wherein at least one functional group is bound to a terminal carbon;
b) contacting a support material with the slurry;
c) maintaining contact of the support material in the slurry for sufficient time for silver to be deposited on the support material;
d) removing liquid from the slurry to form dry solid particles; and
e) calcining the solid particles.

### DETAILED DESCRIPTION OF THE INVENTION

The present invention is for a process of making a silver-containing catalyst which can be used for the direct oxidation of an alkene, such as propylene, to an alkene oxide, such as propylene oxide. The process utilizes an organic compound to form a slurry with a silver compound and water. “Slurry” does not mean a phase separation between the liquid and solid, i.e., a suspension, and does not mean a homogeneous mixture in a single phase in which the solid is dissolved in the liquid, i.e., a solution. “Slurry” means a mixture of insoluble or low soluble solid in liquid. This slurry is contacted with an alkaline earth carbonate. The liquid of the slurry is removed by any means known in the art, such as heating, filtration, evaporation, spray drying, etc. The resulting solid residue is dried and calcined.

Prior art methods of preparing a silver-containing catalyst for the oxidation of an alkene to an epoxide include forming a solution containing a silver compound and a slurry of the solution and a support material. It had been believed that good dispersion of the silver in a solution was required to have good deposition of the silver on the support material. In the present invention, a slurry is formed containing the silver compound before contact with the support material. Organic compounds which if used in greater quantities would be solvents for the silver compound and would form a solution may be used in embodiments the present invention in lesser amounts to form a slurry with the silver compound. These organic compounds are not believed to function primarily as solvents in the present invention but may act as surfactants to promote deposition of the silver onto the support.

The silver compound is not believed to be critical to the present invention. Any silver compound which will form a slurry while not reacting with the organic compound to form an undesirable byproduct and which may be reduced to metallic silver may be used. Examples of silver compounds which may be used in the present invention are silver oxide or a silver salt, such as nitrate, carbonate or carboxylate, such as acetate, propionate, butyrate, oxalate, malonate, malate, maleate, lactate, citrate, phthalate and fatty acid ester and combinations thereof. The silver concentration in the finished catalyst is at least a catalytically effective amount, preferably from about 2 percent to 80 percent by weight, more preferably from about 10 percent to 70 percent by weight, most preferably from about 30 percent to 70 percent by weight and specifically about 54% by weight.

The organic compound has at least one functional group of the formula —NX₂, —OX or [(═O) (—OX)] wherein X is hydrogen or an alkyl of one to three carbon atoms and wherein at least one functional group is bound to a terminal carbon. The functional group [(═O)(—OX)] and the terminal carbon form a carboxyl-type group. The organic compound may be any hydrocarbon, such as an alkane or alkene which may have substituents other than the functional group(s). Functional groups may be bound to carbons other than a terminal carbon as long as at least one functional group is bound to a terminal carbon. One embodiment of the invention includes an organic compound of an alkyl of two to four carbon atoms having at least one functional group of the formula —NX₂, —OX or [(═O) (—OX)] wherein X is hydrogen and wherein one or more functional group is bound to a terminal carbon. Specific embodiments of the organic compound have functional groups of —NH₂, —OH or [(═O) (—OH)]. Examples of such organic compounds are ethylenediamine, ethanolamine, ethylene glycol, propyl ether, propylene glycol (1,2-propanediol), trimethylene glycol (1,3-propanediol), 1-propanol, glycine, triethanolamine, triethylenediamine and triethylamine, diethylenediamine, malonic acid, propionic acid and citric acid. There may be one or more organic compounds used in the present invention.

Optional promoters such as compounds of alkali metals, other metals or halides may be added to the slurry or may be added to the solid catalyst after reduction. Examples of alkali metals are potassium, sodium, rubidium or cesium. Alkali metals compounds may be salts, preferably carbonates, nitrates or nitrites, most preferably potassium nitrate. Examples of other metal promoters which may also be optionally added as promoters are gold, tungsten, rhenium, molybdenum, fluorine, thallium, yttrium, barium, cerium, cobalt, indium and niobium. These metals may be added as compounds such as oxides, acids, carbonates, sulfates, halides, oxyhalides, hydroxyhalides, hydroxides and sulfides. Examples of halide promoters are fluorine or chlorine which can be added as compounds such as silver fluoride or silver chloride. The halide promoter may be omitted if the feedstream of the process of direct oxidation of an alkane contains a halide compound.

Any of these promoters (halide, alkali metal or other metals) may be added with the silver compound or the support, after the support has been added, after contact between the slurry and the support, drying or calcination. The promoters are present in the catalyst in the amount of from about 0.01 to 5% by weight. The alkali metal is preferably present in the amount of from about 2 to 5% by weight, more preferably about 3% by weight. The halide is preferably present in the amount from about 0.01 to 1.0% by weight, more preferably 0.05 to 0.5% by weight. The other metals are preferably present in the amount from about 0.01 to 5.0% by weight, preferably from about 0.1 to 2% by weight.

Embodiments of the invention may have the temperature at which the slurry is formed ranging from about 10° C. to about 90° C. Other embodiments may maintain the temperature at about 20° C. to about 70° C. A specific embodiment is to maintain the temperature at about 50° C. while forming the slurry and contacting the support material in the slurry for deposition of the silver on the support material.

The support material should be an inert solid which is chemically unreactive with the catalytic components. Embodiments of the support material are alkaline earth carbonates, alkaline earth oxides and mixtures thereof.

The alkaline earth carbonate support is of the general formula ACO₃ where A is any Group IIA element, such as beryllium, magnesium, calcium, strontium, barium or radium. One embodiment of the invention uses calcium carbonate. Surface area is preferably from about 5 m²/g to about 25 m²/g.

The alkaline earth oxide support is of the general formula BO where B is beryllium, magnesium, calcium, strontium, barium or radium. One embodiment of the invention uses calcium oxide. The alkaline earth carbonate support may be used with the alkaline earth oxide support or alone. A and B may be different or the same.

The support material is contacted with the slurry formed from the silver compound, water, one or more organic compound(s) and, optionally, one or more promoters. The support material is preferably maintained in contact with the slurry for sufficient time for silver to be deposited on the support. One embodiment for the contact time of the present invention is from 0 to 24 hours. Another embodiment is from 1 to 16 hours. A specific embodiment is for one hour. This contact time may be referred to as aging.

Liquid is removed from the slurry by heating, filtration, evaporation or centrifuge to obtain solid particles of the catalyst precursor. The liquid may be removed simultaneously with drying of the solid particle of the catalyst precursor by such mean as spray drying. The liquid may be evaporated at a temperature of 75° to 150° C.

The solid particles of catalyst precursor may be dried in air or an inert gas at room or elevated temperature. Drying time may be from one hour to twenty-four hours, preferably one to four hours. Drying temperature may be from 110° C. to 250° C., preferably about 250° C. Drying is most preferably for four hours at 250° C. Removing the liquid and drying the solid particles of catalyst precursor may be done in two stages at 750 to 125° C. for at least one hour and 1000 to 150° C. for at least one hour. Drying may be in conjunction with or as preparation for calcination.

The solid particles of catalyst precursor may be sieved or formed by techniques known in the art to obtain desired size and shape.

The catalyst precursor must be calcined to further dry the catalyst and support, react the components, e.g., reduce the silver compound to metallic silver, and remove volatile compounds to have an effective catalyst for epoxidation of an alkene to an alkene oxide. Calcination should be at a temperature of from about 100° C. to about 500° C. for a time of from about one hour to about four hours. Calcination may be in one stage or multiple stages. For example, calcination may be at a temperature of about 250° C. for six hours or at a temperature of 110° C. for one hours and then increasing the temperature by 5 C °/min to a temperature of 300° C. for additional calcination for four hours. Calcination is preferably at a temperature of 300° C. for a time of four hours. A reducing agent, such as hydrogen, may also be used during calcination. Without the present invention and its claims being limited by theory, it is believed that exposing the catalyst to these temperatures during calcination reduces the silver to its elemental form but that while other components (alkali metal carbonate, alkali metals, other metals or halides) may react during calcination they are not reduced to their elemental form.

The catalyst can be brought into contact with an alkene, such as propylene, and oxygen under reaction conditions to selectively convert the alkene to an alkene oxide, such as propylene oxide.

The invention having been generally described, the following examples are given as particular embodiments of the invention and to demonstrate the practice and advantages thereof. It is understood that the examples are given by way of illustration and are not intended to limit the specification or the claims to follow in any manner.

### EXAMPLE 1

Deionized water (40 ml) was added to a beaker (100 ml) with a stir bar and a temperature probe. Potassium nitrate (2.4 g) was added to the beaker and stirred till completely dissolved. Silver oxide (17.8 g) was added to the beaker and stirred for 10 minutes. Ethanolamine (1.8 ml) was added to the beaker, heated to 50° C., and stirred at 50° C. for 10 minutes. A slurry mixture was formed.

Separately, calcium carbonate (11.7 g, Mallinckrodt, #4052) was added to a ball-mill jar. The slurry was poured from the beaker into the ball-mill jar. The slurry and calcium carbonate were mixed well using a Teflon rod, and allowed to sit for 1 hour before calcination. Calcination was carried out in a muffle furnace in air by drying at 110° C. for 1 hour and 130° C. for 1 hour, calcining at 300° C. for 3 hours, and naturally cooling inside the furnace with heating off.

The nominal composition of the catalyst is 54 wt. % Ag, and 3 wt. % K on CaCO3.

After the calcination, the catalyst was crushed and ground to fine powder, and then pressed and sieved to 30-50 mesh prior to the use in epoxidation reaction of propylene to propylene oxide. 5 ml of the freshly prepared catalyst was charged into a straight stainless steel tube reactor. The catalyst was supported by a smaller stainless steel tube insert. Quartz wool was used at both top of bottom of the catalyst. Reaction temperature was measured by a thermocouple via a thermal well inserted into catalyst. Feed stream contains 10 vol. % propylene, 15.5% vol. % oxygen, 10 vol. % carbon dioxide, 200 ppm ethylene chloride, 50 ppm nitric oxide, and balance methane at a total GHSV of 1200 h⁻¹, and a total pressure of 40 psig. The reaction temperature was varied between 220 and 260° C. to achieve about 10% propylene conversion. A GC coupled with a Porapaq Q column was used to analyze feed and products, and methane was also used as an internal standard. After 50 hours on stream, propylene oxide selectivity was 58.6% and propylene conversion 10.2% at 226° C.

### EXAMPLE 2

### Different EA Concentration and Add KNO3 after Calcination

The procedure of Example 1 was followed, except 3.6 ml of ethanolamine was used during the slurry preparation. Potassium nitrate was added after calcination of a catalyst precursor. The catalyst precursor was crushed and ground into fine powder that was added to a round flask. Separately, KNO3 (2.24 g) was first dissolved in 40 ml of deionized water in a beaker, poured into the flask, and well mixed with the catalyst precursor. The mixture was dried in a rota-vap at 70° C. under vacuum for about 30 minutes, and further dried in a muffle furnace at 250° C. for 4 hours. After the drying, the catalyst was ground to fine powder, and then pressed and sieved to 30-50 meshes prior to the use in epoxidation reaction of propylene to propylene oxide. The nominal composition of the catalyst is 54 wt. % Ag, and 3 wt. % K on CaCO3.

Catalyst testing was also performed under the conditions as specified in Example 1. After about 20 hours on stream, propylene oxide selectivity was 57.8% and propylene conversion 10.1% at 226° C.

### COMPARATIVE EXAMPLE 1

### No Organic Compound

The procedure of Example 2 was followed, except no organic compound was used. No slurry form of the mixture of deionized water, potassium nitrate and silver oxide was observed. Silver oxide and water were clearly separated in two phases. Catalyst testing was also performed under the conditions as specified in Example 1. After about 20 hours on stream, propylene oxide selectivity was 45.8% and propylene conversion 7.4% at 264° C.

### EXAMPLES 3-7

### Varying EA Concentration

The procedure of Example 1 was followed, except different amounts of ethanolamine were used during the slurry preparation. The results are presented in Table 1:

TABLE 1

| | Ethanolamine | Temperature | Conversion | Selectivity |
|---|---|---|---|---|
| Example | (ml) | (C.) | (%) | (%) |
| 1 | 1.8 | 226 | 10.2 | 58.6 |
| 3 | 0.10 | 242 | 9.6 | 54.5 |
| 4 | 0.45 | 237 | 9.8 | 58.9 |
| 5 | 0.90 | 229 | 9.6 | 59.0 |
| 6 | 2.70 | 243 | 10.1 | 57.2 |
| 7 | 3.60 | 246 | 10.2 | 50.2 |

### EXAMPLES 8-26, COMPARATIVE EXAMPLES 2-3

### Substitution of EA by Other Organic Compounds

The procedure of Example 1 was followed, except different organic compounds in place of ethanolamine were used during the slurry preparation. Catalyst testing was performed under the conditions as specified in Example 1. After about 20 hours on stream, propylene conversion and selectivity to PO were obtained and are shown in Table 2.

TABLE 2

| | Organic Compound | | | Conversion | |
|---|---|---|---|---|---|
| Example | Name | Amount | Temp. (C.) | (%) | Selectivity (%) |
| 8 | Triethanolamine | 1.2 ml | 229 | 11.0 | 57.3 |
| 9 | Ethylenediamine | 1.0 ml | 244 | 10.0 | 56.6 |
| 10 | Ethylenediamine | 1.8 ml | 238 | 10.0 | 56.4 |
| 11 | Ethylenediamine | 3.6 ml | 244 | 10.1 | 59.8 |
| 12 | Diethylenediamine | 3.5 g | 223 | 9.8 | 56.4 |
| 13 | Triethylenediamine | 4.9 g | 247 | 7.5 | 53.4 |
| 14 | Triethylamine | 3.6 ml | 249 | 9.3 | 52.9 |
| 15 | Ethylene glycol | 1.8 ml | 257 | 9.6 | 50.5 |
| 16 | EDTA | 3.1 g | 231 | 10.4 | 56.5 |
| 17 | Ethylene glycol | 3.6 ml | 226 | 10.6 | 58.2 |
| 18 | 1-Propanol | 3.6 ml | 233 | 13.6 | 57.1 |
| 19 | 1,2-Propylene glycol | 3.6 ml | 228 | 10.1 | 58.4 |
| 20 | 1,3-Propylene glycol | 3.6 ml | 229 | 10.6 | 56.8 |
| 21 | Triethyleneglycol | 3.6 ml | 231 | 10.4 | 56.8 |
| 22 | Glycine | 4.2 ml | 226 | 10.0 | 58.2 |
| 23 | Citric acid | 4.0 g | 229 | 11.0 | 51.1 |
| 24 | Malonic acid | 5.9 g | 235 | 10.3 | 55.2 |
| 25 | Propionic acid | 3.6 ml | 250 | 10.8 | 50.9 |
| 26 | Propyl ether | 3.6 ml | 253 | 10.8 | 55.3 |
| Comp. 2 | Methyl Ethyl Ketone | 3.6 ml | 256 | 3.9 | 52.5 |
| Comp. 3 | 2-Propanol | 3.6 ml | 232 | 3.3 | 51.4 |

EDTA—(Ethylenediamine)tetraacetic acid

Methyl ethyl ketone does not have a functional group of the present invention. 2-propanol has a functional group of the present invention but not on a terminal carbon.

### EXAMPLES 27-32

### Two Organic Compounds

The procedure of Example 1 was followed, but two organic compounds were used, instead of ethanolamine only, during the slurry preparation. The results are shown in Table 3.

TABLE 3

| | Organic Compounds | | | | |
|---|---|---|---|---|---|
| Example | 1 | 2 | Temp. (° C.) | Conversion (%) | Selectivity (%) |
| 27 | 0.5 ml EA | 0.50 ml EG | 224 | 10.2 | 60.0 |
| 28 | 0.5 ml EA | 1.0 ml EG | 233 | 10.9 | 55.0 |
| 29 | 1.0 ml EA | 0.50 ml EG | 229 | 10.7 | 57.0 |
| 30 | 1.0 ml EA | 1.0 ml EG | 230 | 10.5 | 56.4 |
| 31 | 1.8 ml EN | 1.8 ml EG | 228 | 9.9 | 57.3 |
| 32 | 3.6 ml EN | 3.6 ml EG | 233 | 10.6 | 56.9 |

EA—Ethanolamine,
EN—Ethylenediamine,
EG—Ethylene glycol

### EXAMPLES 33-36

### Silver Sources

The procedure of Example 2 was followed, except different silver sources and 3.6 ml of ethanolamine were used during the slurry preparation. The results are shown in Table 5.

TABLE 5

| | | Temperature | Conversion | Selectivity |
|---|---|---|---|---|
| Example | Silver source | (° C.) | (%) | (%) |
| 33 | Silver Oxide | 228 | 9.9 | 57.2 |
| 34 | Silver Oxalate | 232 | 9.9 | 55.8 |
| 35 | Silver Carbonate | 232 | 9.9 | 54.5 |
| 36 | Silver Acetate | 261 | 8.2 | 43.3 |

### EXAMPLES 37-44

### Varying Silver Concentration

The procedure of Example 2 was followed, except different amounts of silver were used during the slurry preparation. The results are shown in Table 6.

TABLE 6

| | Wt % Ag in | Temperature | Conversion | Selectivity |
|---|---|---|---|---|
| Example | Final Catalyst | (° C.) | (%) | (%) |
| 37 | 3 | 270 | 2.8 | 20.7 |
| 38 | 12 | 267 | 9.9 | 42.6 |
| 39 | 27 | 250 | 10.9 | 48.1 |
| 40 | 40 | 234 | 10.2 | 51.7 |
| 41 | 54 | 226 | 10.2 | 57.8 |
| 42 | 70 | 231 | 10.2 | 52.6 |
| 43 | 85 | 260 | 6.2 | 45.6 |
| 44 | 92.2 | 243 | 1.5 | 44.9 |

### EXAMPLES 45-48

### Varying Potassium Nitrate Concentration

The procedure of Example 2 was followed, except different amounts of potassium nitrate were used during the slurry preparation. The results are shown in Table 7.

TABLE 7

| | | Temperature | Conversion | Selectivity |
|---|---|---|---|---|
| Catalyst | Wt % KNO3 | (° C.) | (%) | (%) |
| 45 | 0.3 | 240 | 10.0 | 35.9 |
| 46 | 1.5 | 228 | 10.0 | 52.9 |
| 47 | 3.0 | 228 | 9.9 | 58.2 |
| 48 | 6.0 | 262 | 2.3 | 41.9 |

Obviously, numerous modifications and variations of the present invention are possible in light of the above teachings. It is therefore to be understood that within the scope of the appended claims, the invention may be practiced otherwise than as specifically described herein.

1. A process for making a catalyst for oxidation of an alkene to an alkene oxide comprising:
a) forming a slurry of a silver compound, water and one or more organic compounds having at least one functional group of the formula —NX₂, —OX or [(═O) (—OX)] wherein X is hydrogen or an alkyl of one to three carbon atoms, X being the same or different, and wherein at least one functional group is bound to a terminal carbon;
b) contacting a support material with the slurry;
c) maintaining contact of the support material in the slurry for sufficient time for silver to be deposited on the support material;
d) removing liquid from the slurry to form dry solid particles; and
e) calcining the solid particles.

2. The process of claim 1 wherein the silver compound is an oxide, a salt or carboxylate.

3. The process of claim 2 wherein the silver compound is silver oxide, silver nitrate, silver carbonate, silver acetate, silver propionate, silver butyrate, silver oxalate, silver malonate, silver malate, silver, maleate, silver lactate, silver citrate or silver phthalate.

4. The process of claim 1 additionally comprising adding alkali metal salts in step a) or step b) or after step b), step c), step d) or step e).

5. The process of claim 4 wherein the alkali metals are chosen from the group consisting of potassium, sodium, rubidium and cesium.

6. The process of claim 4 wherein the alkali metal salts are carbonates, nitrates or nitrites.

7. The process of claim 4 wherein the alkali metal salt is potassium nitrate.

8. The process of claim 1 additionally comprising adding a halide compound in step a) or step b) or after step b), step c), step d) or step e).

9. The process of claim 8 wherein the halide compound is silver chloride.

10. The process of claim 1 additionally comprising adding an oxide, acid, carbonate, sulfate, halide, oxyhalide, hydroxyhalide, hydroxide or sulfide of gold, tungsten, rhenium, molybdenum, fluorine, thallium, yttrium, barium, cerium, cobalt, indium or niobium in step a) or step b) or after step b), step c), step d) or step e).

11. The process of claim 1 wherein the support material is an alkaline earth carbonate, an alkaline earth oxide and mixtures thereof.

12. The process of claim 1 wherein the support material is an alkaline earth carbonate of the formula ACO₃ wherein A is any Group IIA element.

13. The process of claim 1 wherein the support material is calcium carbonate.

14. The process of claim 1 wherein the contact time is from 0 to 24 hours.

15. The process of claim 14 wherein the contact time is from 1 to 16 hours.

16. The process of claim 15 wherein the contact time is for one hour.

17. The process of claim 1 wherein the liquid is removed by heating, filtration, evaporation or spray drying.

18. The process of claim 1 wherein the liquid is removed by drying in air or an inert gas.

19. The process of claim 18 wherein drying is for one hour to twenty-four hours at a temperature from 110° C. to 250° C.

20. The process of claim 19 wherein drying is for four hours at 250° C.

21. The process of claim 1 wherein calcining is at a temperature of from about 100° C. to about 500° C. for a time of from about one hour to about four hours.

22. The process of claim 21 wherein the temperature is about 250° C. for six hours.

23. The process of claim 22 wherein the temperature is 110° C. for one hour and then increased by 5 C °/min to a temperature of 300° C. for additional calcination for four hours.

24. The process of claim 1 wherein the functional group is —NH₂, —OH or —OOH.

25. The process of claim 1 wherein the organic compound additionally comprises a functional group of the formula —NX₂, —OX or [(═O)(—OX)] wherein X is hydrogen or an alkyl of one to three carbon atoms, X being the same or different, and wherein the functional group is bound to a carbon other than a terminal carbon.

26. The process of claim 1 wherein the organic compound is ethylenediamine, ethanolamine, ethylene glycol, propyl ether, propylene glycol (1,2-propanediol), trimethylene glycol (1,3-propanediol), 1-propanol, glycine, triethanolamine, triethylenediamine, triethylamine, diethylenediamine, malonic acid, propionic acid or citric acid.

27. The process of claim 1 wherein two organic compounds are selected from the group consisting of ethylenediamine, ethanolamine, ethylene glycol, propyl ether, propylene glycol (1,2-propanediol), trimethylene glycol (1,3-propanediol), 1-propanol, glycine, triethanolamine, triethylenediamine, triethylamine, diethylenediamine, malonic acid, propionic acid and citric acid.

28. The process of claim 27 wherein two organic compounds are selected from group consisting of ethylenediamine, ethanolamine and ethylene glycol.

29. The process of claim 28 wherein one organic compound is selected from group consisting of ethylenediamine and ethanolamine and the other organic compound is ethylene glycol.

30. The process of claim 1 wherein one organic compound is selected from the group consisting of ethylenediamine, ethanolamine, ethylene glycol, propylene glycol (1,2-propanediol), trimethylene glycol (1,3-propanediol), 1-propanol, glycine, triethanolamine, diethylenediamine and malonic acid.

31. The process of claim 1 wherein the silver concentration in the catalyst is 2 percent to 80 percent by weight.

32. The process of claim 31 wherein the silver concentration is from about 10 percent to 70 percent by weight.

33. The process of claim 32 wherein the silver concentration is from about 30 percent to 70 percent by weight.

34. The process of claim 33 wherein the silver concentration is about 54% by weight.

35. The process of claim 1 wherein the alkali metal is present in the catalyst in the amount of from about 0.01 to 5% by weight.

36. The process of claim 35 wherein the alkali metal is present in the amount of from about 2 to 5% by weight.

37. The process of claim 36 wherein the alkali metal is present in the amount of about 3% by weight.
